(19) United States
(12) Patent Application Publication
(10) Pub. No.: US 2025/0042964 A1
(43) Pub. Date: Feb. 6, 2025

(54) METHOD OF TREATING NASH USING A LONG-ACTING MUTANT HUMAN FIBROBLAST GROWTH FACTOR

(71) Applicant: TASLY BIOPHARMACEUTICALS CO., LTD., Shanghai (CN)

(72) Inventors: Jian LI, Tianjin (CN); Jun HAN, Tianjin (CN); Xiaohui MA, Tianjin (CN); Ping TAI, Tianjin (CN); Genbei WANG, Tianjin (CN); Xiaodan CAO, Tianjin (CN); Ruijing HUANG, Tianjin (CN); Yongjie JIN, Tianjin (CN); Jing LI, Tianjin (CN); Chen CHEN, Tianjin (CN); Guoyong JIA, Tianjin (CN); Yuanyuan WANG, Tianjin (CN)

(73) Assignee: TASLY BIOPHARMACEUTICALS CO., LTD., Shanghai (CN)

(21) Appl. No.: 18/920,806

(22) Filed: Oct. 18, 2024

Related U.S. Application Data
(63) Continuation of application No. 16/520,053, filed on Jul. 23, 2019, now Pat. No. 12,152,059.
Application No. 16/520,053 is a continuation-in-part of application No. PCT/CN2018/079482, filed on Mar. 19, 2018.

(30) Foreign Application Priority Data
Mar. 22, 2017 (CN) 201710172824.0

(51) Int. Cl. C07K 14/50; A61K 38/00; A61K 38/18; A61P 1/16

(52) U.S. Cl. CPC C07K 14/50; A61P 1/16; A61K 38/00; A61K 38/1825

(57) ABSTRACT

The invention relates to a method of treatment comprising administering a long-acting mutant human fibroblast growth factor to a subject in need thereof. The said long-acting mutant human fibroblast growth factor is mPBG-CH₂-N^{α}H-mFGF21, wherein mFGF21 consists of SEQ ID NO:1, and the said new use consists of a method of treating non-alcoholic steatohepatitis.

### EARLIER FILED APPLICATIONS

The present application is a continuation in part of PCT/CN2018/079482, filed Mar. 19, 2018, which claims priority to CN201710172824.0, filed Mar. 22, 2017, which are herein incorporated by reference in their entirety.

### TECHNICAL FIELD OF THE INVENTION

The present invention relates to the field of biomedicine, and particularly relates to the use of a long-acting mutant human fibroblast growth factor-21 in in a method of treating non-alcoholic steatohepatitis.

### BACKGROUND ART

Non-alcoholic fatty liver disease (NAFLD), also known as non-alcoholic fatty liver, is caused by a variety of reasons. Lesions of this disease mainly occur in the hepatic lobule characterized by steatosis of hepatic parenchymal cells and triglyceride (TG) accumulation (fat content in liver tissues accounting for over 5% of liver wet weight or more than 1/3 hepatic cells presenting steatosis confirmed by histology). NAFLD is similar to alcoholic liver disease (ALD) in terms of pathological changes except absence of history of excessive drinking (equivalent to ethanol consumption, male <140g/week, female <70g/week) and other clinical pathological syndromes caused by specific liver-damaging factors. NAFLD and NASH have become the leading causes of liver disease in Western countries in the past 20 years. Without timely control, NAFLD can easily progress to NASH as fat continues to accumulate excessively. In addition to excessive accumulation of fat, NASH differs from NAFLD in the presence of inflammatory cell infiltration in liver, the degree of hepatic fibrosis, and the degree of liver cell damage. Furthermore, if NASH continues to progress without effective control, it is highly likely to cause hepatic fibrosis, cirrhosis and even liver cancer.

At present, there are no such drugs for NASH that can be clinically used for long term, safe and effective.

Fibroblast growth factor-21 (FGF-21) is another metabolic regulator recently discovered in vivo.

It belongs to FGF family and specifically acts on liver, fat, and islet cells. FGF-21 can effectively and safely regulate blood glucose and blood fat independently of insulin, which has got researchers' a lot of attention. It has also been reported that FGF-21 can effectively prevent and treat NAFLD induced in vitro (Liu Min et al., Effect of fibroblast growth factor-21 on lipid metabolism of non-alcoholic fatty liver cell model induced in vitro. Journal of Jilin University, May 2012, Vol. 38, No. 3, 477-481.)

CN103193878A, hereby incorporated by reference in its entirety, discloses a new long-acting mutant human FGF and the polyethylene glycol (PEG) conjugate thereof. The protein structure of the said FGF-PEG conjugate consists of an amino acid sequence as follows.

```
(SEQ ID NO: 1)
His Pro Ile Pro Asp Ser Ser Pro Leu Leu Gln Phe Gly Gly Gln Val
1 5 10 15
Arg Gln Arg Tyr Leu Tyr Thr Asp Asp Ala Gln Gln Thr Glu Ala His
                        20 25 30
Leu Glu Ile Arg Glu Asp Gly Thr Val Gly Gly Ala Ala Asp Gln Ser
                35 40 45
Pro Glu Ser Leu Leu Gln Leu Lys Ala Leu Lys Pro Gly Val Ile Gln
        50 55 60
Ile Leu Gly Val Lys Thr Ser Arg Phe Leu Cys Gln Arg Pro Asp Gly
65 70 75 80
Ala Leu Tyr Gly Ser Leu His Phe Asp Pro Glu Ala Cys Ser Phe Arg
                                85 90 95
Glu Leu Leu Leu Glu Asp Gly Tyr Asn Val Tyr Gln Ser Glu Ala His
                        100 105 110
Gly Leu Pro Leu Arg Leu Pro Gln Lys Asp Ser Pro Asn Gln Asp Ala
                115 120 125
Thr Ser Trp Gly Pro Val Arg Phe Leu Pro Met Pro Gly Leu Leu His
        130 135 140
Glu Pro Gln Asp Gln Ala Gly Phe Leu Pro Pro Glu Pro Pro Asp Val
145 150 155 160
Gly Ser Ser Asp Pro Leu Ser Met Val Gly Pro Ser Gln Gly Arg Ser
                                165 170 175
Pro Ser Tyr Ala Ser
                        180
```

Its preparation method is shown in Embodiment 4. This conjugate was later named by its inventors as FG (PEGylation Recombinant Human-mouse Chimeric Fibroblast Growth Factor 21 or mPEG-CH₂-N^{α}H-mFGF21).

FG has the functions of regulating blood glucose, lowering blood triglycerides, and regulating total cholesterol, et al. . . . However, there have been no reports of FG in the treatment of NASH so far.

### DESCRIPTION OF THE INVENTION

Disclosed herein the use of a long-acting mutant human FGF in the preparation of drugs for treating NASH.

As used herein, the long-acting mutant human FGF refers to mPEG-CH₂-N^{α}H-mFGF21 (FG) or a salt.

As used herein, the use includes that FG or its salt can lower the levels of alanine aminotransferase (ALT) and aspartate aminotransferase (AST) in serum, improve steatosis and lobular inflammation, reduce the degree of hepatocellular ballooning degeneration, and improve liver damage.

As used herein, the NASH includes, not limited to, hepatitis-induced NASH, obesity-induced NASH, diabetes-induced NASH, insulin resistance-induced NASH, hypertriglyceridemia-induced NASH, abetalipoproteinemia-induced NASH, NASH induced by glycogen storage disease, NASH induced by Wake's Disease, NASH induced by Wolman's disease, and lipodystrophia-induced NASH.

As used herein, the drugs mean pharmaceutical compositions containing FG or a pharmaceutically acceptable salt thereof as the active pharmaceutical ingredient. The pharmaceutical compositions can be prepared into any pharmaceutically acceptable dosage form, including tablets, capsules, granules, pills, powders, paste, sublimed preparation, dustpowders, solutions, injections, suppositories, sprays, drops, patches, and drop pills. Drugs of the present invention are preferably prepared into injectable drugs, such as powder injections or liquid injections. The liquid injections include water injections, organic solvent injections, and suspension injections, et al.

The preparations of the pharmaceutical compositions for oral administration may contain conventional excipients such as the bonding agent, stuffing bulking agent, diluting agent, tablet compressing agent, lubricating agent, disintegrating agent, coloring agent, flavoring agent, and wetting agent. The suitable stuffing bulking agents include starch, sucrose, cellulose, mannitol, lactose, and other similar ones. The suitable disintegrating agents include starch, polyvinyl pyrrolidone and starch derivatives such as sodium starch glycolate. The suitable lubricating agents include, for example, magnesium stearate. The solid form of the compositions for oral administration can be prepared through conventional methods including mixing, filling, tablet compressing, et al. Repeated mixing allows the active pharmaceutical substance to be distributed throughout the compositions containing a large amount of stuffing bulking agent. The excipients commonly-used include mannitol, sorbitol, sodium pyrosulfite, sodium hydrogen sulfite, sodium thiosulfate, cysteine hydrochloride, thioglycolic acid, methionine, vitamin C, disodium EDTA, calcium disodium edetate, carbonates of alkali metal and aqueous solutions thereof, acetates of alkali metal and aqueous solutions thereof, phosphates of alkali metal and aqueous solutions thereof, hydrochloric acid, acetic acid, sulfuric acid, phosphoric acid, amino acid, sodium chloride, potassium chloride, sodium lactate, xylitol, maltose, glucose, fructose, dextran, glycine, starch, sucrose, lactose, silicon derivatives, cellulose and its derivatives, alginate, gelatin, polyvinyl pyrrolidone, glycerol, Tween 80, agar, calcium carbonate, calcium bicarbonate, surfactant, polyethylene glycol, cyclodextrin, β-cyclodextrin, phospholipids, kaolin, pulvistalci, calcium stearate, magnesium stearate, et al . . .

The usage and dosage of the pharmaceutical composition of the present invention are determined according to conditions of diseases while being used. For example, the pharmaceutical composition can be administered 1-6 times a day for 1-10 doses each, and each dose can be 0.1 mg-1000 mg.

The new use of FG provided by the present invention has the following advantages:

FG can significantly lower the levels of ALT and AST in serum, improve steatosis and lobular inflammation, reduce the degree of hepatocellular ballooning degeneration, and improve the pathological score of liver damage. The final results demonstrate that the present invention can be used to treat NASH with superior effectiveness to prior art.

### DETAILED DESCRIPTION OF IMPLEMENTATION

The present invention will now be further demonstrated using the following embodiments.

For the preparation method of FG used in the present invention, see Embodiment 4 of CN103193878A.

### Embodiment 1: Preparation of Water Injections

1. Composition: FG protein (concentration: 10 mg/mL), histidine (pharmaceutical grade, concentration: 10 mg/mL), citric acid-sodium citrate buffer (20 mM sodium citrate-citric acid, 100 mM NaCl, pH5.5±0.1).

2. Preparation method 1) 12.5 mg/mL FG protein solution, the buffer system is citric acid-sodium citrate, and the pH is adjusted to 5.5±0.1, prepared for use:

2) 50 mg/mL histidine mother liquor, the buffer system is citric acid-sodium citrate, the pH is adjusted to pH 5.5±0.1, prepared for use:

3) Mix the FG protein solution of step 1) and the histidine mother liquor of step 2) according to the volume ratio of 4:1, and transfer 500uL of the mixed solution into 2mL penicillin bottles.

### Experiment Example 1: Therapeutic Effect of FG in an Effective Dose Range in a Mouse Model of MCD (Methionine Choline Deficient) Diet-Induced NASH.

A mouse model of MCD diet-induced NASH is used in this experiment, mainly because this diet has been used for more than 40 years and its production process has become more mature. In addition, the characterization this diet induced has obtained multiple verification in terms of its rapidity (i.e. NASH related symptoms can be triggered in about 4 weeks) and effectiveness (i.e. symptoms induced have great similarity to those of human NASH).

1. Experimental method

After adaptive feeding for two weeks, C57BL male mice of eight-week-old were began to be fed MCD diet. After MCD diet fed for two weeks, the mice were randomly divided into 4 groups according to the body weight: a solvent group, a FGF-21 group, a low-dose FG group, and a high-dose FG group. There were 10 mice in each group and they were administered once a day via hypodermic injection for two weeks.

Details of animal grouping and administration are shown in Table 1:

TABLE 1
Animal grouping and administration

| | | | | Administration |
|---|---|---|---|---|
| Group | n | Dose | | period (day) |
| Solvent group | 10 | Solvent in the same | | 14 d |
| | | volume | | |
| FGF-21 group | 10 | 0.4 | mg/kg/d | 14 d |
| Low-dose FG group | 10 | 0.125 | mg/kg/d | 14 d |
| High-dose FG group | 10 | 2 | mg/kg/d | 14 d |

After the administration, the mice were killed and their relevant tissues were removed for subsequent analyses.

2. Indices detection

2.1 The blood serum was separated and various biochemical indices therein were detected:

levels of ALT and AST in serum were determined using a clinically general assay kit (provided by Shanghai Shensuo-UNF Medical. Diagnostic Articles Co., Ltd).

2.2 Histomorphological examination on mouse liver tissue

Morphological changes in mouse liver were observed through H&E staining method. The specific steps were as follows: The freshly removed small piece of liver was fixed in formalin solution overnight, and embedded in paraffin after gradient dehydration, then sectioned into 5um slices. The liver sections were stained with hematoxylin-eosin, and finally the morphology of liver tissue of each mouse was observed under a microscope.

2.3 Detection of collagen fibers in mouse liver tissue

The collagen deposition in mouse liver was observed through Sirius Red (SR) staining method. The specific steps were as follows: The liver sections from paraffin blocks were stained using the Sirius Red staining kit, and finally the fibrosis of liver tissue of each mouse was observed under a microscope.

There is a scoring system for assessing the severity of NASH in clinical practice, i.e., NASH

Clinical Research Network Scores, mainly including three aspects: steatosis, lobular inflammation, and ballooning degeneration. In this experiment, five mice were randomly selected from each group, and five microscopic fields of view within the H&E stained liver tissue sections of the corresponding mouse were randomly selected based on which a score is given from the above three aspects, objectively evaluating the degree of hepatitis of mice in each group.

3. SPSS16.0 statistical software was used for statistics and data analysis. A t-test was used to compare the difference between two groups of data. One-way ANOVA test and multiple linear regression analysis were used to compare differences among multiple groups of data. The difference was statistically significant with P<0.05.

4. Experimental results

4.1 Effect of FG on serum ALT level as shown in Table 2:

TABLE 2
Effect of FG on serum ALT level

| | Solvent | FGF-21 | Low-dose FG | High-dose FG |
|---|---|---|---|---|
| | group | group | group | group |
| Serum | 199.8 ± 30.8 | 100.2 ± 20.6* | 51.1 ± 7.7***Δ | 39.9 ± 4.1***Δ |
| ALT(U/L) | | | | |

(1) Data shown in the table represented the mean ± standard error;
(2) *compared with the solvent group P < 0.05; ***compared with the solvent group P < 0.001; Δcompared with the FGF-21 group P < 0.05.

As shown in Table 2, after two weeks of treatment, the ALT level of mice with NASH in the FGF-21group decreased to 100.2±20.6 U/L, and there was a significant difference compared with the solvent group with the ALT level 199.8±30.8 U/L (P<0.05). The levels of ALT in the low-dose FG group and high-dose FG group decreased to 51.1±7.7 U/L and 39.9±4.1 U/L, respectively, and a significant difference was found compared with the solvent group (P<0.001) as well as the FGF-21 group (P<0.05). These results indicate that FG not only has the efficacy of lowering ALT levels but also its efficacy herein is better than FGF-21.

4.2 Effect of FG on serum AST level as shown in Table 3:

TABLE 3
Effect of FG on serum AST level

| | Solvent | FGF-21 | Low-dose FG | High-dose FG |
|---|---|---|---|---|
| | group | group | group | group |
| Serum | 196.9 ± 29.3 | 115.7 ± 15.9* | 74.4 ± 7.9**^{Δ} | 73.7 ± 7.8**^{Δ} |
| ALT(U/L) | | | | |

(1) Data shown in the table represented the mean ± standard error;
(2) *compared with the solvent group P < 0.05; **compared with the solvent group P < 0.01; ^{Δ}compared with the FGF-21 group P < 0.05.

As shown in Table 3, after two weeks of treatment, the AST level of mice with NASH in the FGF-21group decreased to 115.7±15.9U/L, and there was a significant difference compared with the solvent group with the AST level 196.9±29.3 U/L (P<0.05). The levels of AST in the low-dose FG group and high-dose FG group decreased to 74.4±7.9U/L and 73.7±7.8U/L, respectively, and a significant difference was found compared with the solvent group (P<0.01) as well as the FGF-21 group (P<0.05). These results indicate that FG not only has the efficacy of lowering ALT levels but also its efficacy herein is better than FGF-21.

4.3 Effect of improvement on NASH clinical scores as shown in Table 4:

TABLE 4
Effect of improvement on NASH clinical scores

| | | Lobular | | |
|---|---|---|---|---|
| | Steatosis | inflammation | Ballooning | Total score |
| Solvent group | 2.2 ± 0.2 | 2.6 ± 0.1 | 1.3 ± 0.1 | 6.0 ± 0.21 |
| FGF-21 group | 2.0 ± 0.3 | 2.1 ± 0.2 | 0.9 ± 0.1** | 4.7 ± 0.5* |
| Low-dose FG | 1.8 ± 0.3 | 0.7 ± 0.1***^{ΔΔΔ} | 0.7 ± 0.1** | 3.2 ± 0.4***^{Δ} |
| group | | | | |
| High-dose FG | 0.9 ± 0.3**^{Δ} | 0.6 ± 0.2***^{ΔΔΔ} | 0.4 ± 0.1***^{ΔΔ} | 1.9 ± 0.5***^{ΔΔ} |
| group | | | | |

(1) Data shown in the table represented the mean ± standard error;
(2) *compared with the solvent group P < 0.05; **compared with the solvent group P < 0.01; ***compared with the solvent group P < 0.001; ^{Δ}compared with the FGF-21 group P < 0.05; ^{ΔΔ}compared with the FGF-21 group P < 0.01; ^{ΔΔΔ}compared with the FGF-21 group P < 0.001.

As shown in Table 4, after two weeks of treatment, both the scores for ballooning and NASH total scores in FGF-21 group had improved significantly (P<0.05) compared with the solvent group, whereas no significant improvement in scores for lobular inflammation had been observed (P>0.05). Compared with the solvent group, there was a significant improvement in both low-dose FG group and high-dose FG group in terms of scores for lobular inflammation, scores for ballooning and NASH total scores. Scores for steatosis in high-dose FG group had been significantly improved compared with the solvent group. In addition, in comparison with FGF-21 group, all scores for steatosis, lobular inflammation, ballooning and the total thereof had been significantly improved in low-dose FG group and/or high-dose FG group. These results indicate that FG not only has the efficacy of improving lobular inflammation, ballooning, and NASH total scores but also its efficacy herein is better than FGF-21.

5. Experimental results

Detections after injection of FG show that compared with the solvent group and FGF-21 group, low-dose FG group and/or high-dose FG group can significantly lower the levels of ALT and AST in serum, improve steatosis and lobular inflammation, reduce the degree of hepatocellular ballooning degeneration. and improve the pathological score of liver damage. These results indicate that FG has the effect of treating NASH.

6. Conclusion: FG has the clinical application value in the treatment of NASH which is superior to the prior art.

1-13. (canceled)

14. A pharmaceutical composition comprising mPEG-CH2-NαH-mFGF21 or a pharmaceutically acceptable salt thereof.

15. The pharmaceutical composition of claim 14, wherein the mFGF21 consists of the amino acid sequence set forth in SEQ ID NO: 1.

16. The pharmaceutical composition of claim 14, wherein the molecular weight of mPEG-CH2-NαH-mFGF21 is about 55 kD.

17. The pharmaceutical composition of claim 14, further comprising a citrate buffer.

18. The pharmaceutical composition of claim 14, wherein the pharmaceutical composition has an acidic pH.

19. The pharmaceutical composition of claim 14, wherein the pharmaceutical composition comprises about 10 mg/mL mPEG-CH2-NαH-mFGF21.

20. A method of treating non-alcoholic steatohepatitis (NASH) in a subject, comprising administering to the subject the pharmaceutical composition of claim 14, wherein the method improves steatosis and lobular inflammation.

21. The method of claim 20, wherein the NASH is selected from the group consisting of hepatitis-induced NASH, obesity-induced NASH, diabetes-induced NASH, insulin resistance-induced NASH, hypertriglyceridemia-induced NASH, abetalipoproteinemia-induced NASH, NASH induced by glycogen storage disease, NASH induced by Wake's Disease, NASH induced by Wolman's disease, and lipodystrophia-induced NASH.

22. The method of claim 20, wherein the pharmaceutical composition is administered to the subject subcutaneously.

23. The method of claim 20, wherein the pharmaceutical composition is a pharmaceutically acceptable dosage form.

24. The method of claim 23, wherein the dosage form is selected from the group consisting of tablets, capsules, granules, pills, powders, paste, sublimed preparation, dust powders, solutions, powder injection, liquid injections, suppositories, sprays, drops, patches, and drop pills.

25. The method of claim 24, wherein the liquid injections are selected from the group consisting of water injections, organic solvent injections, and suspension injections.

26. The method of claim 20, wherein the pharmaceutical composition is administered to the subject at a dose of at least about 0.125 mg/kg.

27. The method of claim 20, wherein the pharmaceutical composition is administered to the subject at a dose of up to about 2 mg/kg.

28. The method of claim 20, wherein the pharmaceutical composition is administered to the subject at a dose of between about 0.125 mg/kg and about 2 mg/kg.

29. A method of treating non-alcoholic steatohepatitis (NASH) in a subject, comprising administering to the subject the pharmaceutical composition of claim 14, wherein the method lowers levels of alanine aminotransferase or levels of aspartate aminotransferase in serum.

30. The method of claim 29, wherein the NASH is selected from the group consisting of hepatitis-induced NASH, obesity-induced NASH, diabetes-induced NASH, insulin resistance-induced NASH, hypertriglyceridemia-induced NASH, abetalipoproteinemia-induced NASH, NASH induced by glycogen storage disease, NASH induced by Wake's Disease, NASH induced by Wolman's disease, and lipodystrophia-induced NASH.

31. The method of claim 29, wherein the pharmaceutical composition is administered to the subject subcutaneously.

32. The method of claim 29, wherein the pharmaceutical composition is a pharmaceutically acceptable dosage form. 33 (New) The method of claim 32, wherein the dosage form is selected from the group consisting of tablets, capsules, granules, pills, powders, paste, sublimed preparation, dust powders, solutions, powder injection, liquid injections, suppositories, sprays, drops, patches, and drop pills.

34. The method of claim 33, wherein the liquid injections are selected from the group consisting of water injections, organic solvent injections, and suspension injections.

35. The method of claim 29, wherein the pharmaceutical composition is administered to the subject at a dose of at least about 0.125 mg/kg.

36. The method of claim 29, wherein the pharmaceutical composition is administered to the subject at a dose of up to about 2 mg/kg.

37. The method of claim 29, wherein the pharmaceutical composition is administered to the subject at a dose of between about 0.125 mg/kg and about 2 mg/kg.

38. A method of treating non-alcoholic steatohepatitis (NASH) in a subject, comprising administering to the subject the pharmaceutical composition of claim 1, wherein the method improves steatosis and lobular inflammation, and wherein the method reduces the degree of hepatocellular ballooning degeneration.

39. The method of claim 38, wherein the NASH is selected from the group consisting of hepatitis-induced NASH, obesity-induced NASH, diabetes-induced NASH, insulin resistance-induced NASH, hypertriglyceridemia-induced NASH, abetalipoproteinemia-induced NASH, NASH induced by glycogen storage disease, NASH induced by Wake's Disease, NASH induced by Wolman's disease, and lipodystrophia-induced NASH.

40. The method of claim 38, wherein the pharmaceutical composition is administered to the subject subcutaneously.

41. The method of claim 38, wherein the pharmaceutical composition is a pharmaceutically acceptable dosage form.

42. The method of claim 41, wherein the dosage form is selected from the group consisting of tablets, capsules, granules, pills, powders, paste, sublimed preparation, dust powders, solutions, powder injection, liquid injections, suppositories, sprays, drops, patches, and drop pills.

43. The method of claim 42, wherein the liquid injections are selected from the group consisting of water injections, organic solvent injections, and suspension injections.

44. The method of claim 38, wherein the pharmaceutical composition is administered to the subject at a dose of at least about 0.125 mg/kg.

45. The method of claim 38, wherein the pharmaceutical composition is administered to the subject at a dose of up to about 2 mg/kg.

46. The method of claim 38, wherein the pharmaceutical composition is administered to the subject at a dose of between about 0.125 mg/kg and about 2 mg/kg.


SEQUENCE LISTING (1 sequence)

SEQ ID NO: 1 - protein, 181 aa, synthetic construct
  source 1..181: note = Description of Artificial Sequence: Synthetic polypeptide
HPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLRLPQKDSPNQDATSWGPVRFLPMPGLLHEPQDQAGFLPPEPPDVGSSDPLSMVGPSQGRSPSYAS